(19) United States
(12) Patent Application Publication
(10) Pub. No.: US 2009/0258347 A1
(43) Pub. Date: Oct. 15, 2009

(54) METHOD FOR DIAGNOSING AND MONITORING CELLULAR RESERVOIRS OF DISEASE

(75) Inventor: Lesley Erica Scott, Randburg (ZA)

Correspondence Address: RATNERPRESTIA, P.O. BOX 980, VALLEY FORGE, PA, 19482, US

(73) Assignee: UNIVERSITY OF THE WITWATERSRAND, Johannesburg (ZA)

(21) Appl. No.: 11/721,760

(22) PCT Filed: Dec. 12, 2005
(86) PCT No.: PCT/IB2005/003738

§ 371 Date: Jun. 14, 2007

(30) Foreign Application Priority Data
Dec. 14, 2004 (ZA) 2004/10087

(51) Int. Cl. C12Q 1/68

(52) U.S. Cl. 435/6

(57) ABSTRACT

The invention provides an assay for diagnosing and/or monitoring a viral infection or disease in a patient, the assay including the steps of mixing a sample of leucocytes with a fluorescent cell membrane-permeable dye which stains RNA or both DNA and RNA within the leucocytes; identifying from all the leucocytes at least two of the three major sub-populations of leucoytes selected from the group consisting of monocytes, granulocytes and lymphocytes; determining the fluorescence intensity for each of the identified sub-populations; and comparing the fluorescence intensity of at least two cell sub-populations to each other to obtain at least one of the following ratios: monocytes:granulocytes, monocytes:lymphocytes, and granulocytes lymphocytes. The viral infection may be HIV and the disease may be AIDS. The invention also provides a method of monitoring the cellular viral, parasitic or bacterial reservoir of a patient with a viral or bacterial infection by the steps described above. A, kit for performing the assay or method is also provided.

### BACKGROUND OF THE INVENTION

CD4 monitoring and HIV viral load measurement in HIV disease are the bedrock to monitoring quality-care of HIV infected patients. In HIV disease, viral load is one of the best markers of dynamic changes over time. The viral load is principal to facilitate prediction about disease progression, predict response to therapy and monitor the effects of that therapy. The viral load assays currently quantitate across a wide range of viral load levels (linear dynamic range), and have good reproducibility of 0.2 log. Quantitative measurements of plasma HIV RNA are expressed in two ways: the number of HIV-RNA copies/ml of plasma (or IU/ml), or the logarithmic equivalent (log₁₀, where a 1-log change represents a 10-fold change). A 3-fold variation (0.5log₁₀ copies) is accounted for by intra-assay variability and biological variability, but clinically a 10 fold (1-log₁₀) difference is regarded as significant.

The laboratory measure of HIV plasma viral load is performed by nucleic acid amplification techniques that amplify a target region of DNA or RNA. It is an extremely sensitive and skilled laboratory tool that requires a dedicated laboratory environment with skilled staff that adhere to strict protocol to prevent carry over contamination. This methodology is also expensive and dependent on the supply of expensive kits and equipment for testing. Currently there are three FDA licensed HIV RNA assays accepted for clinical management—reverse transcriptase PCR Roche Amplicor HIV-1 Monitor™ Test, bioMerieux NucliSens® HIV-1 QT Assay, and Versant® HIV-1 RNA 3.0 Assay (bDNA). All three assays are high throughput, the Amplicor and the NASBA assays amplify the target HIV-RNA into measurable amounts of nucleic acid product (target amplification), whereas the bDNA amplifies the signal obtained from a captured HIV-RNA target (signal amplification).

The cost of a single viral load test (Roche Amplicor) ranges from about US$50-US$100. This is either unaffordable or unavailable (not feasible from an implementation perspective for high throughput testing) in the developing world, especially for patient follow-up. Several alternative cost effective methodologies are being investigated that use different platforms. For example, the p24 antigen quantitation ELISA assay (Perkin-Elmer Life and Analytical Sciences, Turku, Finland) is becoming increasingly popular as an inexpensive alternative that measures viral replication in vivo by quantitating the major viral core protein-p24 The measure of viral reverse transciptase activity recovered from plasma and measured in an ELISA format by the ExaVir™ enzyme immunoassay (Cavidi Tech-AB, Uppsala, Sweden) has also been developed as an alternative cost effective assay.

Several other factors have been shown to correlate with disease progression, and form the basis of other approaches to laboratory diagnostic monitoring tools under exploration. A few examples of these factors are:
(i) serum levels of soluble urokinase-type plasminogen activator receptor that is shown to be an independent predictor of survival in HIV;
(ii) soluble immune factors such as:
   plasma levels of β₂ microglobulin;
   tumour necrosis factor type II;
   soluble CD27 that positively correlate with each other, and sCD27 that is a good independent marker of CD4⁺ T cell decline in HIV infection;
   soluble CD40 ligand in HIV infection is shown to serve as a new surrogate marker to assess treatment efficacy;
   levels of soluble CD8 are also shown to correlate with CD38 expression in asymptomatic HIV infection;
   neopterin produced by human monocyte/macrophages upon stimulation, has been suggested as a marker in HIV and other autoimmune diseases to
   measure the extent of cellular immune activation and the extent of oxidative stress;
   levels of endothelial markers have also been found to correlate significantly with initial viral load;
   Haemoglobin has also shown to be an independent prognostic indicator of HIV;
   lipid and acute-phase protein alterations in early HIV infection are also found to correlate with disease progression.

In spite of all these alternative approaches, the viral load remains the most important and clinically useful measure for monitoring. Nevertheless, there is still a need for a viral load monitoring assay or alternative disease monitoring assay or test that is affordable, reliable, simple and robust to increase the accessibility to viral load measurement in the developing world.

### SUMMARY OF THE INVENTION

According to a first embodiment of the invention, there is provided an assay for diagnosing and/or monitoring a viral infection or disease, the assay including the steps of:
mixing a sample of leucocytes with a fluorescent cell membrane-permeable dye which stains RNA or both DNA and RNA within the leucocytes;
identifying from all the leucocytes at least two of the three major sub-populations of leucoytes selected from the group consisting of monocytes, granulocytes and lymphocytes;
determining the fluorescence intensity for each of the identified sub-populations; and
comparing the fluorescence intensity of at least two cell sub-populations to each other.

The sample of leucocytes may be from a blood sample of a patient (which includes a cord blood sample), in which case the assay may also include the step of lysing the red blood cells so as to obtain the leucocyte sample. Alternatively, cultured cells may form the leucocyte sample.

Typically, the monocyte, granulocyte and lymphocyte sub-populations are all identified in the assay.

The fluorescence intensity of each sub-population may be determined from the mean or median fluorescence intensity or from marker or region limits of the respective sub-population.

Typical ratios that may be calculated by comparing the fluorescence intensity of one to sub-population to the fluorescence intensity of another sub-population are: monocytes:granulocytes, monocytes:lymphocytes and granulocytes:lymphocytes.

The ratio of the mean fluorescence intensity of the monocyte population to the mean fluorescence intensity of the granulocyte population or lymphocyte population may be an indicator of the cellular viral reservoir in the patient.

The viral infection may be HIV. Similarly, the disease may be AIDS. For example, when monitoring HIV/AIDS infection, the monocyte:granulocyte ratio will be greater than one and is expected to increase with increase of the virus reservoir. However, the ratio of these two sub-populations or the ratio of a different combination of two of the leucocyte sub-populations may vary when monitoring a different disease, such as tuberculosis.

The assay may also be used to monitor co-infection of the patient with another disease, for example, another viral, parasitic or bacterial infection For example, if the ratio of the mean fluorescence intensity of the monocyte population to the mean fluorescence intensity of the granulocyte population is less than the ratio of the mean fluorescence intensity of the monocyte population to the mean fluorescence intensity of the lymphocyte population, this may be an indicator of a co-infection, such as Mycobacteium tuberculosis infection. This relationship may similarly be shown by the mean fluorescence intensity of the granulocyte to lymphocyte population being either <1 (showing lymphocyte activity/disease) or >1 (showing granulocyte activity/disease).

The dye is preferably a compound which stains RNA or both DNA and RNA. The dye may be selected from the group consisting of thiazole orange, SYTO dyes, LDS-751 and acridine orange.

May be performed using a flow cytometer, haematology analyser or other suitable instrumentation that measures fluorescence, such as a fluorimeter.

The assay may also include a step for obtaining a CD4 count. In particular, an antibody that fluoresces in a different fluorescent channel to the dye may be added to the sample so that the CD4 count can be obtained. Other antibody markers may also be used, for example cell activation markers such as CD38 or specific sub-population markers such as CD14 and CD16 or p24.

According to a second embodiment of the invention, there is provided a method of diagnosing and/or monitoring the cellular viral reservoir (load) of a patient with HIV or other bacterial infection, the method including the step of comparing the mean fluorescence intensity of the patient's monocytes that have been stained with a fluorescent dye to the mean fluorescence intensity of the patient's granulocytes and/or lymphocytes that have also been stained with a fluorescent dye.

This comparison may be used as a marker of the viral load of the patient, and hence as a marker of disease infection or progression and related infections, as well as being used to indicate the patient's response to therapy.

According to a third embodiment of the invention, there is provided a kit for performing the assay described above, the kit including a cell membrane-permeable dye which stains RNA or both DNA and RNA, typically but not necessarily with a single fluorescence.

The kit may further include a set of computer readable instructions for performing the assay or at least a portion of the assay, and in particular, for:
identifying at least two of the monocyte, granulocyte and/or lymphocyte sub-populations;
calculating a fluorescence intensity of each identified sub-population; and/or
comparing the fluorescence intensity of one sub-population to another to obtain at least one ratio.

The computer readable instructions may further interpret the ratio or ratios obtained above. For example, the computer readable instructions may indicate to a user whether the patient has a low, medium or high virus reservoir or has a co-infection.

The fluorescence intensities of each sub-population may be the mean or median fluorescence intensity or may be a region or marker limit of that sub-population.

The kit may further include an antibody for determining the CD4 count (or other cell marker) of the sample.

The kit may further include one or more reagents selected from the group consisting of a red cell lysing agent, a stabilizer, a fixative, control cells, media and bead reagents.

The kit may further include means for dispensing the red cell lysing agent, dye, antibody reagents and/or other reagents used in the assay.

The kit may further include other sets of cell membrane markers or intracellular markers for phenotyping, such as CD38, CD14/CD16 or p24.

According to a further embodiment of the invention, there is provided a machine readable medium comprising instructions for diagnosing or monitoring a viral infection or disease according to the method of the invention, which when executed by a machine, cause the machine to perform all or at least some of the steps of the assay described above.

The machine readable medium may be configured for use in conjunction with a flow cytometer and/or haematology analyser.

The machine readable medium may include instructions for performing analysis methods selected from the group consisting of impedance, light scatter and fluorescence.

### BRIEF DESCRIPTION OF THE DRAWINGS

FIG. 1 shows (b) a histogram of thiazole orange used to identify leucocytes from an HIV⁻ specimen, and (a) a dot plot showing the lymphocytes with lowest side scatter (described as complexity on the vertical axis) followed by monocytes and granulocytes with the most SSC (side scatter).

FIG. 2 shows histograms of thiazole orange used to identify leucocytes from three HIV⁺ specimens (d), (e) and (f), and corresponding dot plots (a), (b) and (c), respectively, showing the monocytes (in region ‘C’) with increased FL1 fluorescence by a right shift from the reference line through the background cell populations (lymphocytes and granulocytes, ‘B’ and ‘D’).

FIG. 3 shows an example of the HIV reservoir monitoring index (HIV^{rmi}) (the name given to this test) determined according to the invention versus log plasma viral load determined according to the Roche Amplicor method, from patients on ARV (Antiretroviral).

FIG. 4 shows two graphs illustrating the correlation between CD4 counts and (b) the HIV reservoir monitoring index (HIV^{rmi}) determined according to the method of the invention and (a) log plasma viral load determined according to the Amplicor method.

FIG. 5 shows the correlation of the HIV reservoir monitoring index (HIV^{rmi}) determined according to the method of the invention and intracellular p24 (shown both as relative fluorescence and percentage cell positivity in monocytes).

FIG. 6 shows dot plots of a leucocyte sample from a patient who is infected with HIV and possibly also tuberculosis (TB). The granulocytes (region B) have increased dye (in this case, thiazole orange) fluorescence in relation to the lymphocytes (region D).

FIG. 7 shows dot plots of an assay according to the invention in which a CD4 count was also generated.

FIG. 8 shows dot plots of an example where in addition to the HIV reservoir monitoring index (HIV^{RMI}) being calculated, CD14/CD16 immunophenotyping was also determined.

FIG. 9 shows a graph of percentage CD14low/CD16high cells of all the monocytes plotted against the highest HIV^{RMI} index value obtained for 14 HIV positive randomly selected specimens.

FIG. 10 shows a graph similar to FIG. 3 of an example of the HIV reservoir monitoring index (HIV^{rmi}) determined according to the invention versus log plasma viral load determined according to the Roche Amplicor method, from HIV⁺ naïve patients. Here the highest HIV^{rmi} value is plotted against the Roche plasma viral load.

FIG. 11 shows a set of graphs of the HIV^{RMI} on three HIV⁺ patients followed longitudinally up to 12 weeks after ARV. The graphs on the left plot the CD4 count, the plasma viral load (as determined by RNA, Amplicor assay) and the HIV^{RMI}. The plots on the right exclude the CD4 count. These plots shows how the HIV^{RMI} is useful in monitoring patients on therapy, where the HIV^{RMI} shows increases or decreases with viral load and or immune reconstitution (measured by the CD4 count).

FIG. 12 shows a graph of the HIV^{RMI} results from a cohort of paediatric patients aged 30 days to 50 days. A cut-off value of HIV^{RMI}=2.0 shows those patients above the line to be confirmed HIV⁺ by the PCR Amplicor assay, and those below the line to be HIV⁻. The dots just show blood samples measured in the assay <10 hours old (▪)and <24 hours old (♦).

FIG. 13 shows a graph of HIV^{RMI} results from a second cohort of paediatric patients with a range in ages. ▪ are HIV⁻ patients as determined by DNA PCR, Amplicor test, and ♦ are HIV⁺ patients confirmed by DNA PCR, Amplicor test. The two graphs are divided into two age categories: (a) <49 days and (b) >50 to <200 days. These plots show the effect that infant age has on the HIV^{RMI} as a qualitative assay for HIV diagnosis in infants.

### DETAILED DESCRIPTION OF THE INVENTION

The invention provides an assay for diagnosing and/or monitoring a viral infection or disease, such as HIV/AIDS.

It has long been documented that the death of CD4 T-cells is an untoward outcome of the viral replicative cycle in these cells. Emerging in the literature is the premise that CD4 T-cells are innocent bystanders and the CD4+ macrophages have a more significant and direct role to play in HIV/AIDS pathogenesis.

Macrophages have been shown to be the principal reservoir of HIV and SHIV (simian immunodeficiency virus/HIV-1 chimera) and sustain high virus loads after the depletion of the CD4 T-cells. The macrophages are infected during the acute infection and the number infected gradually increases over time and become a major contributor to total body virus burden during the symptomatic phase of the disease. Long-term infections of HIV in monocytes have also been shown in patients receiving HAART [1].

It is being recognised that assessing viral load should not be restricted to the plasma RNA, and changes in HIV DNA and RNA copy numbers in peripheral blood mononuclear cells should be given equal focus. In particular, HIV-1 mRNA expression in peripheral blood cells has been shown to predict disease progression independently of the CD4 count [2].

The applicant thus set out to investigate whether it would be possible to monitor viral load by quantifying the cellular nucleic acid in leucocytes. HIV/AIDS was chosen for testing as a suitable example of a viral infection and disease, as there is a pressing need for an affordable and reliable viral monitoring assay for this disease.

It has now been found that monocytes of HIV positive patients contain an increased amount of nucleic acids, and this increase correlates to the plasma viral load. Furthermore, the applicant has found that by quantifying the cellular (whole cell) nucleic acid (RNA or both DNA and RNA) using a fluorescent dye, and comparing the amount of nucleic acids in the monocytes with the amount of nucleic acids in the granulocytes (neutrophils) and in the lymphocytes, it is possible to monitor the cellular viral reservoir load. More particularly, the applicant has shown that the index (ratio) of monocyte, lymphocyte and granulocyte mean fluorescent intensities (MFI) can be used as a marker of HIV/AIDS disease progression and related infections.

As the increased nucleic acid concentration in the monocytes is probably a measure of virus reservoir (or cellular response to infection), the mean fluorescence ratio or index (MFI) calculated according to the invention has been termed the HIV reservoir monitoring index (HIV^{rmi}).

Mycobacterium tuberculosis is the etiological agent for tuberculosis infection. This bacterium is a facultative parasite capable of surviving and multiplying in phagocytes. During primary infection, M.tuberculosis enters and survives in alveolar macrophages, and disseminates from the lung by a heterogeneous group of tissue macrophages. It has also been shown that neutrophils play a role in TB infection as the ‘Trojan horse’ by hiding mycobacteria from the immune system. In addition, neutrophil function has been shown to be impaired in HIV/TB infection, resulting in increased susceptibility to secondary infections. The identification of certain groups of patients from TB cohorts with increased neutrophil fluorescence in the HIV^{rmi} assay provides an additional application of cellular reservoir identification using HV^{rmi}. The hypothesis that the HIV^{RMI} increased neutrophil fluorescence is a measure of intracellular M.tuberculosis infection (or cellular response to infection) is being investigated.

The other infections may also be parasitic infections, such as bilharzia or worms.

Flow cytometry is a platform well-used for measuring antigen expression and cell enumeration. Several studies using this platform have found correlates to HIV disease progression. The flow cytometry platform has also been used to detect and quantitate viruses directly, including HIV, and was therefore decided to be a particularly suitable platform for performing the assay of the invention. It will be apparent to a person skilled in the art, however, that the assay may also be performed on a haematology analyser or by fluorimetry without requiring undue experimentation.

Nucleic acid binding dyes are well described in flow cytometry for discriminating non-nucleated from nucleated cell events in assays that measure cell viability and ploidy analysis. The direct measure of nucleic acid specific dyes on intact cells has, however, been mostly applied to study apoptosis and necrosis, and is relatively uninvestigated for the direct measurement of viral DNA or RNA for viral load measurement.

Suitable dyes for use in the assay should have the following properties:
Whole cell staining
Cell permeate dye (vital staining)
Dye that binds DNA and RNA or RNA only
Dye excitation/emission spectra should be compatible with detection by flow cytometry, haematology analysers and (optionally) fluorimetry.

Some of the commercially available vital probes (permeate) that have been described for use in flow cytometry and that have these properties are thiazole orange, SYTO group dyes (from Molecular Probes), LDS-751, acridine orange and the combination of Hoechst 33342 and pyronin Y (some SYTO dyes, like SYTO RNA Select, which are also cell membrane-permeable but only stain RNA, may also show the same increased fluorescence).

Acridine orange can be used as a vital stain without fixation of the cells, but requires two different excitation sources to visualize DNA and RNA at the same time. The absorption of acridine orange is in the range between 440 nm and 480 nm (blue), and the emission is in the range between 520 nm (green for DNA) and 650 nm (orange for RNA). The combination of Hoechst 33342 and pyronin Y can be used for DNA and RNA content in intact cells, but requires two light sources.

The above examples do not include DNA/RNA binding dyes that are currently used for microscopy, DNA/RNA amplification, and detection molecular methods that have not yet been cited for use in flow cytometry. Although the most popular flow cytometry configurations use 488 nm lasers light sources, there are also other light sources at different wavelengths that would be compatible with different dyes.

Thiazole orange is an asymmetric cyanine that consists of two aromatic rings connected by a bond and is sufficiently soluble in a phosphate buffer or distilled water solution to make appropriate dilutions for long term storage, with negligible fluorescence in solution. The interaction of thiazole orange with nucleic acids is through complex intercalation (insertion of planar compounds between adjacent base pairs) which is dependant on the state of the nucleic acid (single or double stranded) and has higher affinity for A-T rich sequences. Once bound to nucleic acid the thiazole orange aromatic rings become restricted and reduce their rotation, which is believed to cause the intense fluorescence [3]. Thiazole orange is used in flow cytometry to identify Plasmodium parasitized red blood cells, stain RNA in reticulocytes and measure the percentage reticulated platelets within whole blood. Quantities of thiazole orange used for nucleic acid detection are generally in the order of 10⁻⁶ to 10⁻⁷ M free dye and 10⁻⁵ M in applications for flow cytometry.

Thiazole orange is a suitable dye for use in this invention, because it is membrane permeate, it is suitable with standard ‘lyse no wash’ protocols and it has an emission and excitation spectrum similar to FITC (fluoroscein isothiocyanate). It can also be used with standard blue laser light (488 nm) flow cytometers. The commercial cost of thiazole orange is approximately ZAR778.00 (−$80) for 1 gram. Dilutions of thiazole orange to the concentrations required in this assay would result in about 600 tests costing only 1 cent (ZAR0.01). Such minimal expense makes this dye a good candidate for affordable HIV/AIDS monitoring in the developing world.

The assay is typically performed as follows:

A sample of peripheral whole blood in EDTA is prepared and the red cells are lysed. A cell-permeable dye is then added to the remaining white cell suspension and the dye binds to the DNA and RNA within the cells. The bound dye fluoresces, making it possible for the cells in suspension to be analysed for fluorescence and side angle light scatter by flow cytometry (488 nm laser instrument detecting thiazole orange in channel FL1).

Three white cell populations (granulocytes, monocytes, lymphocytes) are identified using a dual scattergram (SSC vs FL1), although it would also be possible to identify only the monocyte population and one of the granulocyte and lymphocyte populations.

The mean fluorescent (FL1) intensity (MFI) in each gated cell type is recorded, and the ratio of monocyte mean fluorescent (FL1) intensity (MFI) to granulocyte MFI the ratio of the monocyte MFI to lymphocyte MFI, and the ratio of granulocyte mean fluorescent (FL1) intensity (MFI) to lymphocyte mean fluorescent (FL1) intensity (MFI) is calculated.

A CD4 count can be determined in the same tube at the same time, by adding an antibody that fluoresces in a different channel to the dye used for the cellular nucleic acids.

This assay is best performed on fresh (<24hrs) blood, since aged blood shows a general increase, throughout all the leucocytes, in thiazole orange mean fluorescent intensity (MFI).

Preliminary investigation into the exact cause of the increased mean fluorescent intensity (MFI) in monocytes indicates that the thiazole orange measures RNA in the cytoplasm. It is also probable that because the dye is used in small molar concentrations, it is just sufficient to enter cells and stain cytoplasmic nucleic acid such as RNA. Increases in DNA due to cell replication in the nucleus may not be able to be measured at these low dye concentrations and therefore not interfere with the MFI measurement. The hypothesis that this increased RNA is viral and/or upregulated mRNA (cellular response to infection) is being further validated, but the role of monocytes in HIV further strengthens this hypothesis.

HIV-1 replication has been shown to continue in patients receiving ARV with suppressed plasma vireamia. Sites of replication are found In cellular reservoirs including monocytes. In particular a specific subgroup of monocytes with the phenotype CD14low/CD16high have been shown to be more susceptible to HIV infection, and to contribute to those monocytes that differentiate into macrophages to traffic the virus through tissue. A preliminary study has shown that the percentage of these CD14low/CD16high monocytes correlates with increasing HIV^{RMI} (highest index value: monocytes to granulocytes or monocytes to lymphocytes in the presence of probable TB co-infection), and further validates HIV^{RMI} as a measure of cellular HIV reservoir. The graph in FIG. 9 shows this correlation (r=0.59) for 14 HIV positive specimens. A good positive correlation exists between the two variables but with only 35% of the data represented by the equation of the line shown in the figure.

The HIV^{RMI} assay was primarily investigated as a monitoring tool for HIV adult patients on ARV. A single HIV^{RMI} result may not be useful for direct conversion (prediction) to a plasma viral load value without knowledge of patient treatment status. The HIV^{RMI} does appear useful for longitudinal monitoring as an early indicator of virus production/cell activity for disease progression and response to therapy. FIG. 11 illustrates how the HIV^{RMI} of a patient shows the correct response to therapy with the CD4 count increasing and the viral load and HIV^{RMI} decreasing. This was present in 22% of an ARV cohort studied. The second patient shows a response in the CD4 count and the HIV^{RMI}, but no change in the plasma viral load. This was present in 50% of the cohort studied. The third patient shows no response in the CD4 count or the plasma viral load, but a response to therapy in the HIV^{RMI}. This was present in 27.7% of the cohort. Changes detected by the HIV^{RMI} not yet reflected in the plasma viral load may explain the non response in the CD4 count.

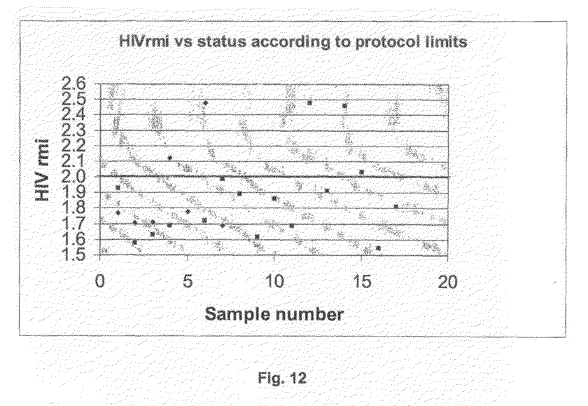

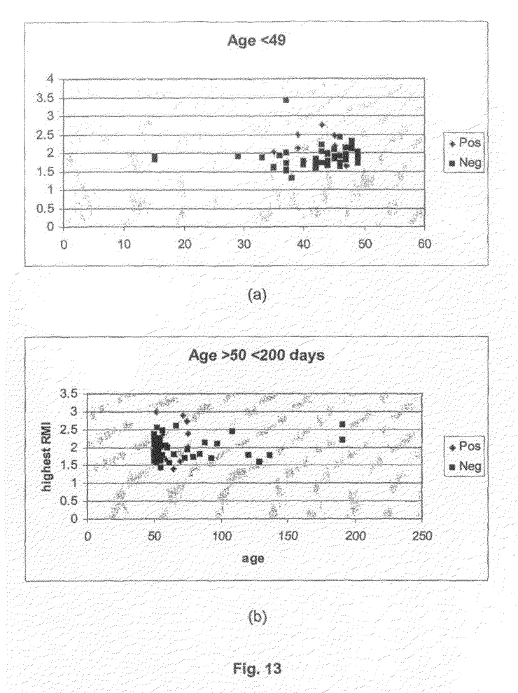

The HIV^{RMI}, however, was also (secondarily) investigated as a qualitative assay for use in diagnosis of HIV. This became apparent when the HIV^{RMI} values measured in a paediatric cohort (infants age 30-50 days old) where found to exceed values typical of adult monitoring values. Applying a cut-off value of HIV^{RMI}=2.0 (FIG. 12), it was shown that the HIV^{RMI} is capable of identifying HIV+ from HIV− samples and shows concordance with PCR HIV DNA Amplicor results. Additional analysis in a different cohort of infants ranging in ages up to 200 days old (FIG. 13) showed that as an infant's immune system matures and becomes activated, the HIV^{RMI} becomes less reliable as a qualitative HIV cut-off, and is then more useful as a monitoring tool.

The fact that early HIV infection may be detected by the HIV^{RMI} (as found in the paediatric cohort) may also mean that the HIV^{RMI} assay may be useful in detecting PHI (primary or acute HIV infection) in adults that are sero-negative and in the <2 week after infection window period. This is being investigated.

It is envisaged that a kit for performing the assay described above can be provided to make it easier for the invention to be performed. The kit would include one or more of the following:
a cell membrane-permeable dye which stains both DNA and RNA, typically with a single fluorescence;
antibodies for determining the CD4 count (or other cell marker) of the sample;
other sets of cell membrane markers or intracellular markers for phenotyping, such as CD14/CD16 or p24;
one or more reagents, such as a red cell lysing agent, a stabilizer, a fixative, control cells, media and bead reagents;
means for dispensing the red cell lysing agent, dye, antibody reagents and/or other reagents used in the assay;
a set of computer readable instructions for performing the assay or at least a portion of the assay, and in particular, for identifying at least two of the monocyte, granulocyte and/or lymphocyte sub-populations; calculating a fluorescence intensity of each identified sub-population; and/or comparing the fluorescence intensity of one sub-population to another to obtain at least one ratio. The computer readable instructions may further interpret the ratio or ratios obtained above. For example, the computer readable instructions may indicate to a user whether the patient has a low, medium or high virus reservoir or has a co-infection.

It is further envisaged that there will be provided a machine readable medium comprising instructions, which when executed by a machine, cause the machine to perform all or at least some of the steps of the invention described above. The machine readable medium may be configured for use in conjunction with a flow cytometer and/or haematology analyser, and may include instructions for performing analysis methods such as impedance, light scatter and fluorescence.

The present invention is further described by the following examples. Such examples, however, are not to be construed as limiting in any way either the spirit or scope of the invention.

### EXAMPLES

Blue plastic tubes (Beckman Coulter, cat #2523749) were labelled with individual laboratory numbers, and 50 μl AB human reagent serum (blood transfusion services) was inserted into each tube as a blocking agent.

Fresh EDTA was mixed with a sample of whole blood from each patient on a blood rocker for 3-5 minutes at room temperature. 50 μl of each EDTA and whole blood sample was added to a tube containing the AB serum, taking care to wipe excess blood off the pipette tip so as to ensure that no blood was deposited onto the sides of the tube. The blood and serum were mixed for 30 seconds and the tubes were incubated for 15 minutes at room temperature. The red cells were then lysed using Immunoprep™ reagent (Beckman Coulter) dispensed by an automated Q-Prep system (Beckman Coulter).

A 10 μM thiazole orange (Sigma/Aldrich, cat #39,006-2) solution in methanol was prepared. 1 μM was diluted in Sorenson's Phosphate Buffer, (pH adjusted to 7.2) or distilled water. A volume of 40 μl of this 1 μM diluted thiazole orange solution was added to each tube after red cell lysis and the tubes were incubated at room temperature for a further 20 minutes in the dark.

The samples were then analysed on an XL MCL (Beckman Coulter) flow cytometer, counting a minimum 25000 leucocyte events. All leucocytes were identified using heterogeneous gating (SSC vs FL1 thiazole orange) in the FL1 channel.

Three regions were set around the granulocytes, monocytes and lymphocytes, and the mean fluorescent intensity (MFI) in the FL1 channel for each region was measured. The ratios of monocyte MFI to granulocyte MFI and monocyte MFI to lymphocyte MFI and granulocytes to lymphocytes was calculated using the following formula, as an example:

ratio (or index value)=Monocyte MFI/Granulocyte MFI or Lymphocyte MFI=Reservoir Monitoring Index (RMI)

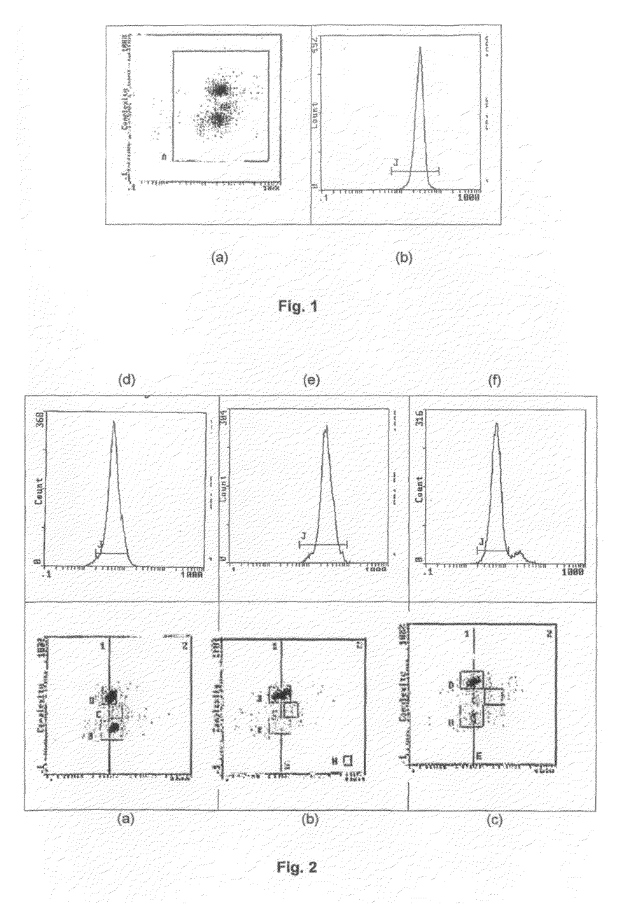

It was found that the leucocytes from an HIV negative sample share similar mean fluorescence intensity (MFI) in the FL1 channel, as shown by the single FL1 histogram in FIG. 1(b). Reproducibility of this assay was found to have a CV (coefficient of variation) of 1.54%. The dot plot of FIG. 1(a) shows the lymphocytes with lowest side scatter (described as complexity on the vertical axis) followed by monocytes and granulocytes with the most SSC.

However, the mean fluorescent intensity ratios in HIV positive patients (FIG. 2) with a reproducibility of 1.13% CV differ to the mean fluorescent intensity ratios in HIV negative patients (FIG. 1). Thiazole orange, used to isolate intact cells of HIV positive samples, was shown to produce a different fluorescent intensity on certain cell populations during HIV infection. The monocytes from HIV positive patients have increased MFI, which is illustrated by the wider spread histograms (d), (e) and (f) of FIG. 2. The dot plots show the monocytes (in region ‘C’) with increased FL1 fluorescence by a right shift from the reference line through the background cell populations (lymphocytes and granulocytes) (FIGS. 2(a), (b) and (c)).

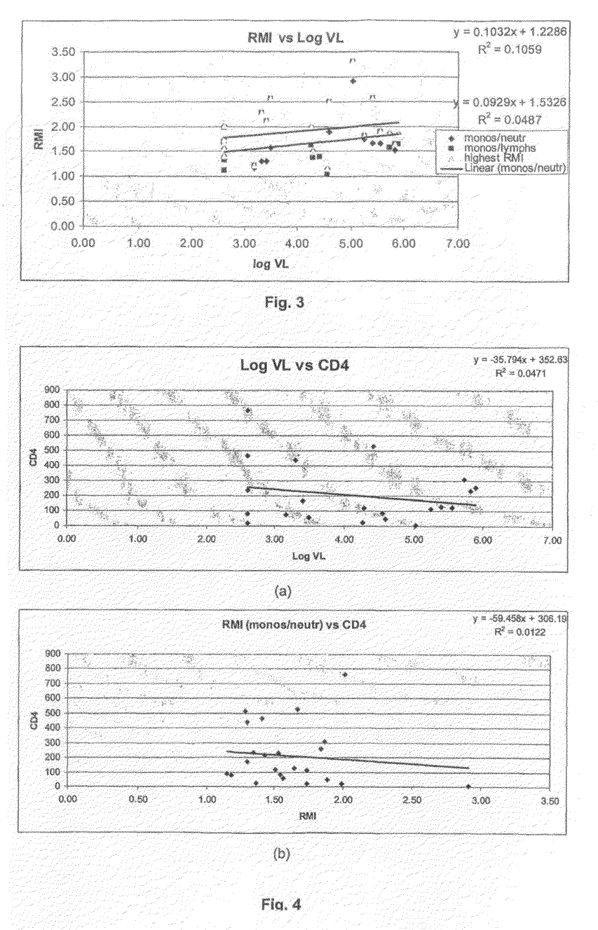

The samples which were assayed as described above were also analysed using the standard Roche Amplicor Monitor version 1.5 assay to determine the log plasma viral load. A positive correlation was shown to exist between the HIV^{RMI} and plasma viral load (VL) (Table 1 and FIG. 3).

Explanation of Table 1:

Value Indicative of Virus Reservoir (Column 4 and 5)

The HIV^{RMI} with the highest value (monocyte/granulocyte or monocyte/lymphocyte) is the index correlated to plasma viral load and indicative of the amount of intracellular viral reservoir or mRNA cellular response to infection.

Value Indicative of Additional Disease (such as TB) (Column 6).

Granulocyte/lymphocyte=1 shows no other background cellular activity;

Granulocyte/lymphocyte <1 shows disease with lymphocyte activity (may be early or late stage lymphocyte infection/activation);

Granulocyte/lymphocyte >1 shows disease with granulocyte activity (possible TB).

The correlation between HIV^{RMI} and plasma PCR viral load on ARV naïve patients was initially shown to be r=0.677 p<0.0001 (R²=0.357, n=80) (Table 3).

The viability of the samples was determined, and only those samples with ≧60% viability (n=80) were included in the study. The highest HIV^{RMI} was compared with the log viral load (Roche Amplicor) (Table 2).

TABLE 1
An example of CD4 counts, plasma viral load and HIV^{rmI} for several randomly selected patients

| | | | | | Additional | |
|---|---|---|---|---|---|---|
| | | | | | disease | |
| | Plasma PCR | | HIV RMI: | HIV RMI | indicator | |
| CD4 count | viral load | | monocytes/ | monocytes/ | granulocytes/ | |
| cells/μl | copies/ml | Log viral load | granulocytes | lymphocytes | lymphocytes | Comments |
| 82 | 400 | 2.6 | 1.55 | 1.34 | 0.86 | with lymphocyte activity |
| 465 | 400 | 2.6 | 1.41 | 1.33 | 0.94 | with lymphocyte activity |
| 310 | 530000 | 5.72 | 1.87 | 1.58 | 0.84 | with lymphocyte activity |
| 22 | 18300 | 4.26 | 1.99 | 1.63 | 0.82 | with lymphocyte activity |
| 259 | 779000 | 5.89 | 1.84 | 1.65 | 0.89 | with lymphocyte activity |
| 231 | 655000 | 5.82 | 1.53 | 1.64 | 1.07 | |
| 439 | 2040 | 3.31 | 1.3 | 2.28 | 1.72 | with high granulocyte activity |
| | | | | | | (possible TB) |
| 59 | 3120 | 3.49 | 1.57 | 2.56 | 1.62 | with high granulocyte activity |
| | | | | | | (possible TB) |
| 169 | 2560 | 3.41 | 1.3 | 2.11 | 1.63 | with high granulocyte activity |
| | | | | | | (possible TB) |
| 48 | 39600 | 4.6 | 1.89 | 2.49 | 1.32 | with granulocyte activity |
| | | | | | | (possible TB) |
| 87 | 35700 | 4.55 | 1.15 | 1.03 | 0.89 | with lymphocyte activity |
| 76 | 1470 | 3.17 | 1.18 | 1.23 | 1.04 | |
| 20 | 400 | 2.6 | 1.74 | 1.13 | 0.64 | with high lymphocyte activity |
| 529 | 26500 | 4.42 | 1.67 | 1.39 | 0.83 | with lymphocyte activity |
| 119 | 19400 | 4.29 | 1.51 | 1.38 | 0.92 | |
| 4 | 107000 | 5.03 | 2.91 | 3.3 | 1.01 | |
| 112 | 177000 | 5.25 | 1.74 | 1.81 | 1.03 | |
| 128 | 251000 | 5.4 | 1.65 | 2.56 | 1.55 | With high granulocyte activity |
| | | | | | | (possible TB) |
| 125 | 354000 | 5.55 | 1.65 | 1.89 | 1.14 | with some granulocyte |
| | | | | | | activity (possible TB) |
| 236 | 400 | 2.6 | 1.35 | 1.68 | 1.25 | with granulocyte activity |
| | | | | | | (possible TB) |
| 765 | 400 | 2.6 | 2.01 | 1.98 | 0.98 | |

TABLE 2

| Variable | Maximum | Mean | Minimum | N | Median |
|---|---|---|---|---|---|
| log VL | 5.88 | 4.04 | 1.70 | 80 | 4.57 |
| Highest RMI | 5.51 | 2.33 | 1.22 | 80 | 1.99 |

Linear regression:

| | |
|---|---|
| R-Square | 0.3568 |

TABLE 3
Spearman Correlation Coefficients, N = 80
Prob > |r| under H0: Rho = 0

| | Highest RMI |
|---|---|
| log VL | 0.677 |
| | <.0001 |

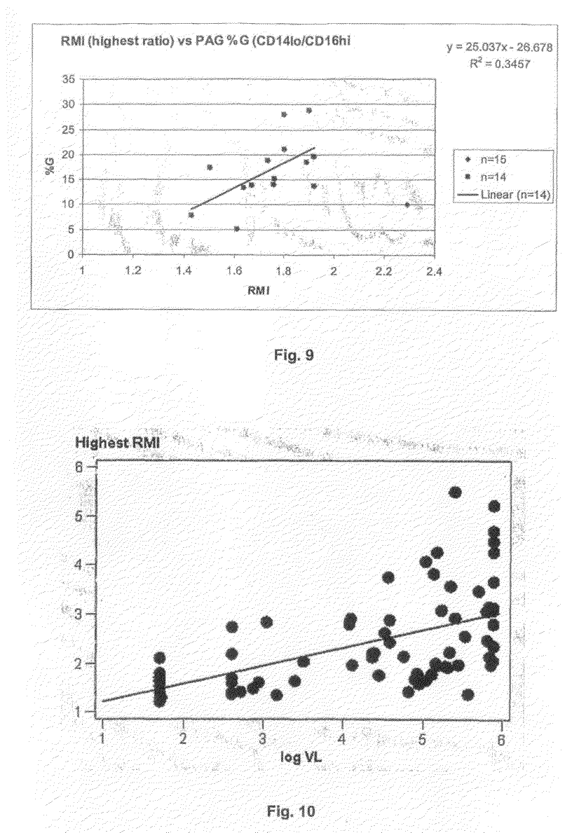

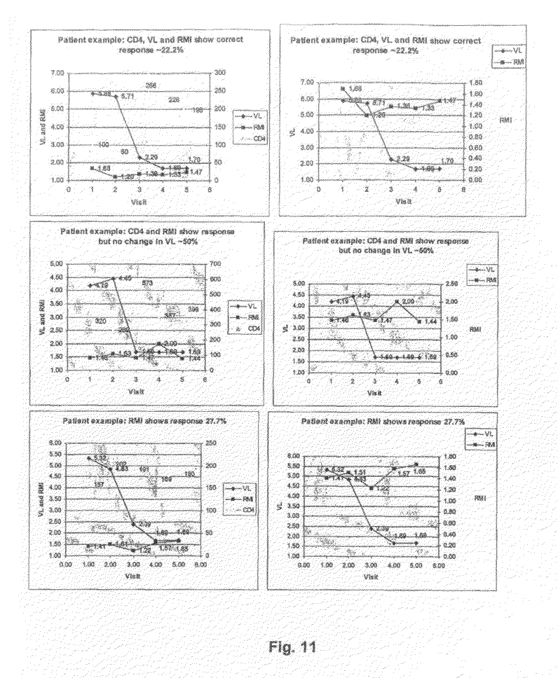

The HIV^{RMI} was shown to significantly correlate with the viral load, although only 35% of the data is represented by the equation of the line in FIG. 10. Limitations are shown with the upper limit of the Roche viral load assay.

However in the era of anti-retrovirals (ARV), this correlation has reduced to r=0.244 (R²=0.106, n=20), as shown in FIG. 3.

Patients receiving anti-retroviral treatment (ARV) will pass through different phases of infection, and will show differences between plasma and cellular viral loads, which is why a single HIV^{RMI} result is not useful for direct conversion to a plasma viral load value without knowledge of the patient treatment status:
Phase 1: Decreased plasma viral load: clearance of free virions from the plasma (t_{1/2}<6 hrs) and decay of short-lived infected CD4 T-lymphocytes (t_{1/2} 1-2 days).
Phase 2: clearance of viral reservoir from infected macrophages and mononuclear cells in lymphoid tissue (t_{1/2} 1-4 weeks).
Phase 3: slow viral decay in latent reservoirs with persistent detection of viral replication .

Samples from some patients also showed an increase in granulocyte fluorescent intensity, resulting in the monocyte/granulocyte index being less than the monocyte/lymphocyte index (highlighted in column 5 of Table 1). These patients were found to be co-infected with tuberculosis and this relationship is thus being investigated as an additional tool (TB^{RMI}) for identification and monitoring of co-infection. This additional index may assist in overall patient monitoring.

FIG. 4 shows the correlation of the HIV^{RMI} and plasma viral load against CD4 counts for naive patients. The negative correlation between the HIV^{RMI} and CD4 count is similar to the negative trend between plasma PCR viral load and CD4 count documented in other studies .

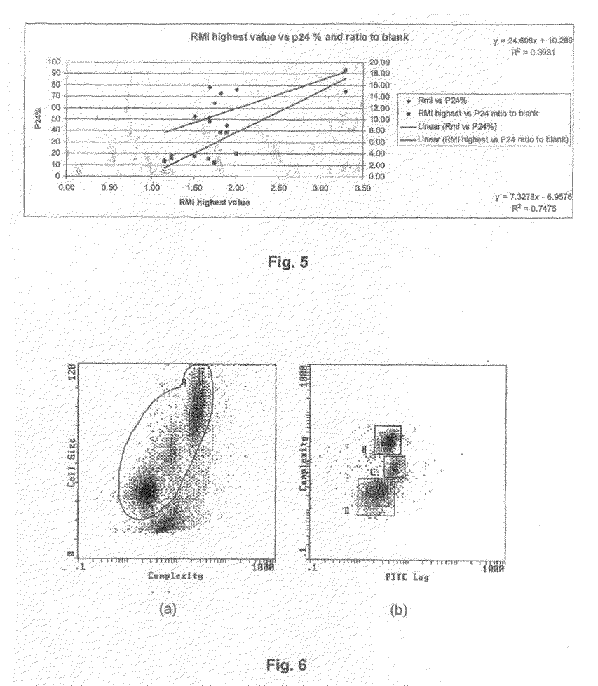

FIG. 5 shows a strong correlation of the HIV^{rmi} with intracellular p24 (viral coat protein), which is also determined by flow cytometery.

FIG. 6 shows a strong correlation between HIV^{rmi} and the percentage monocytes expressing CD14low/CD16high.

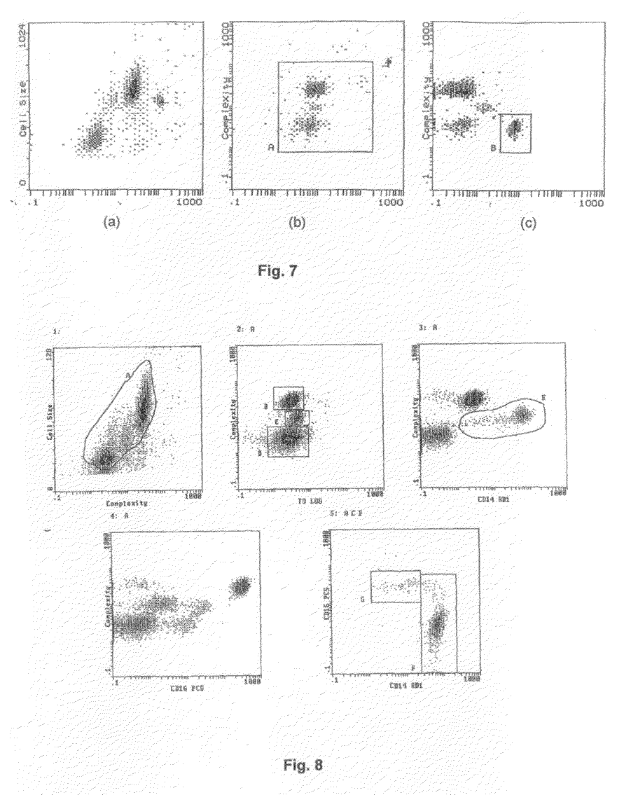

FIG. 7 shows an example using thiazole orange nucleic acid binding dye with CD4 PE (Phycoerythrin) to generate a CD4 count in the same tube as the HIV^{RMI}. The first plot (a) measures light scatter parameters (cell size/forward scatter vs cellular granularity/complexity/side scatter), this plot also contains Flow Count beads (Beckman Coulter) for single platform absolute counting. The second plot (b) measures side scatter vs FL1 thiazole orange fluorescence, the leucocytes are identified in region A. The third plot (c) measures side scatter vs CD4PE fluorescence, with the CD4 lymphocytes identified in region B as a function of all the leucocytes from region A.

FIG. 8 shows dot plots of an example where in addition to the HIV reservoir monitoring index (HIV^{RMI}) being calculated, CD14/CD16 immunophenotyping was also determined. The HIV^{RMI} is calculated from the scatter plot #2, and the percentage CD14low/CD16 high population is calculated from the scatter plot #5, using CD14PE and CD16PC5.

These differences in naïve and ARV patients highlight the strength of the HIV^{RMI} for long term follow-up of cellular reservoirs and not circulating plasma virus that is more readily cleared by ARV. This is shown in FIG. 11, with three patients' longitudinal data shown up to 12 weeks after therapy. The mean viral load for the total group (n=18 patients) in the first five visits was 3.19 (1.69-5.88) c/ml, the HIV^{RMI} 1.52 (1.04-5.27) and the CD4 count 217 (13-573) cells/μl. At baseline, the mean plasma viral load of 4.9 (3.9-5.8) c/ml decreased to 1.9 (1.69-2.6) c/ml at week 8 and remained at 1.9 (1.69-5.1) c/ml to week 12. The mean CD4 count increased from 173 (13-270) cells/μl (baseline) to 243 (48-573) cells/μl at week 4, but remained without change at 245 (72-399) cells/μl to week 12. The mean HIV^{RMI} decreased as the plasma viral load from 1.49(1.2-1.89) at baseline to 1.41(1.19-1.7) at week 4, but increased to 1.45 (1.04-2) at week 8 and 1.76 (1.24-5.27) at week 12. No direct correlation was found between the plasma viral load and the HIV^{RMI} for random samples analysed, irrespective of their treatment status, n=90 (r=0.107, p=0.314). This is due to the HIV^{RMI} increasing where no change in plasma viral load was detected. The CD4 count increased, viral load decreased and the HIV^{RMI} decreased over the visits, as expected in response to therapy, in 22.3% of patients. In 27.7% of patients, the HIV^{RMI} showed increases before any changes occurred in the CD4 count or viral load. In 50% of patients, the HIV^{RMI} increased where a decrease in the CD4 count was detected, with no change in the viral load.

The HIV^{RMI} is also applicable to disease monitoring in paediatric patients as in adults. The HIV^{RMI} values in paeditrics, however, have been noticed to reach higher values than found with adults. Table 4 lists HIV^{RMI} values from a paediatric and an adult cohort.

TABLE 4
HIV^{RMI} values from a paediatric and adult cohort showing higher HIV^{RMI}
values are reached in the paediatric patients.

Highest RMI

Paediatrics:

| | |
|---|---|
| DNA PCR | |
| POS | 2.03 |
| POS | 2.12 |
| POS | 2.48 |
| POS | 1.77 |
| POS | 2.75 |
| POS | 2.46 |
| POS | 2.18 |
| POS | 1.63 |
| NEG | 1.89 |
| NEG | 1.81 |
| NEG | 1.91 |
| NEG | 1.86 |
| NEG | 1.58 |
| NEG | 1.62 |
| NEG | 1.93 |
| NEG | 1.52 |
| NEG | 1.72 |

Adults:

| | |
|---|---|
| Log VL | |
| 5.81 | 1.47 |
| 5.83 | 1.29 |
| 2.31 | 1.41 |
| 2.31 | 1.63 |
| 1.70 | 1.32 |
| 5.15 | 1.31 |
| 5.26 | 1.53 |
| 2.95 | 1.36 |
| 2.40 | 1.62 |
| 1.69 | 1.56 |
| 4.35 | 1.61 |
| 4.30 | 1.57 |
| 1.69 | 1.31 |
| 1.69 | 1.4 |
| 1.69 | 1.79 |
| 4.49 | 1.34 |
| 4.71 | 1.49 |
| NEG | 1.67 |
| NEG | 1.41 |
| NEG | 1.44 |

This concept of paediatrics having higher HIV^{RMI} values was investigated to determine whether the HIV^{RMI} assay could also be used as a qualitative assay for HIV diagnosis. DNA PCR is routinely used for HIV infant diagnosis at 6 weeks of age. When the cut-off of 2.0 for the HIV^{RMI} was applied to a paediatric cohort also tested by DNA PCR, as shown in FIG. 12, it showed concordance with the DNA PCR results. All those specimens with an HIV^{RMI}>2 were DNA PCR positive and all those specimens with an HIV^{RMI}<2 were DNA PCR negative. This was further investigated in a larger cohort (n=132), with infants ranging in ages up to 200 days old (FIG. 13). The HIV^{RMI} showed increased sensitivity (probability that it is positive) and specificity (probability that it is negative) on the younger age group as listed in Table 5.

TABLE 5
Calculations of sensitivity and specificity of the HIV^{RMI} on
a paediatric cohort (n = 132) ranging in age groups.

| An HIV^{RMI} cut-off of 2.0 | Sensitivity | Specificity |
|---|---|---|
| Age 35-49 days (n = 61) | 75% | 81% |
| Age 50-191 days (n = 71) | 54% | 59% |

Several studies have shown difficulty in determining differences in lymphocyte subsets between infected and un-infected infants using immune activation markers, due to changes occurring in the maturation of the infant immune system. This same effect may apply to the HIV^{RMI}, and infants at earlier ages are being investigated, including cord blood.

The inventor believes that the assay according to the invention is advantageous for at least the following reasons:
it is a measure of cellular viral reservoir load and not plasma suspended viral load, and therefore may indicate viral increase sooner than is detectable in the plasma.
it is an overall monitor of disease including other cellular infections such as TB.
the assay is not subtype specific, which is often a concern with PCR methods.
the method of preparation is quick and not labour intensive, with little manipulation of biohazard specimen, especially with the ‘lyse no wash’ protocol (no washing, no extraction).
the result can be reported within 1 hour, which is less than any other known assay for viral measurement.
existing flow cytometric equipment can be used with standard flow cytometric protocols.
only small volumes of blood are required (50 μl/test) and the assay can thus be applied to paediatric specimens.
a CD4 count using thiazole orange assisted PLG (PanLeucogate) (described in more detail in PCT application PCT/IB02/02725, which is incorporated herein in its entirety) can be generated in the same tube. This single tube assay also costs less (˜$4.4) than a standard CD4 count (˜$5.4), since the CD45 mAB reagent is replaced with a much cheaper ‘off-the-shelf’ dye (December 2002).
the assay may be transferred to other smaller platforms with the potential for near patient analysis.
the assay may also be used on a haematology analyser as a general indicator of disease, performed on all routine blood specimens tested for general haematological parameters.
the application of the HIV reservoir monitoring index (HIV^{rmi}) in further research may prove valuable in the involvement of macrophages in this disease and therapeutic monitoring.
the assay may also be useful as a diagnostic tool for HIV in paediatric patients less than 40 days old and in sero-negative adults within 2 weeks of infection.

While the invention has been described in detail with respect to specific embodiments thereof, it will be appreciated by those skilled in the art that various alterations, modifications and other changes may be made to the invention without departing from the spirit and scope of the present invention. It is therefore intended that the claims cover or encompasses all such modifications, alterations and/or changes.

### REFERENCES

1. Lambotte, O., et al., Detection of infectious HIV in circulating monocytes from patients on prolonged highly active antiretroviral therapy. J Acquir Immune Defic Syndr, 2000. 23(2): p. 114-9.
2. Saksela, K, et al., Human immunodeficiency virus type 1 mRNA expression in peripheral blood cells predicts disease progression independently of the numbers of CD4+ lymphocytes. Proc Natl Acad Sci USA, 1994. 91(3): p. 1104-8.
3. Nygren, J., N. Svanvik, and M. Kubista, The interactions between the fluorescent dye thiazole orange and DNA. Biopolymers, 1998. 46(1): p. 39-51.

1.-43. (canceled)

44. An assay for diagnosing or monitoring a viral, parasitic or bacterial infection or disease in a patient, the assay comprising the steps of:
mixing a sample of leucocytes with a fluorescent cell membrane-permeable dye which stains RNA within the leucocytes;
identifying from all the leucocytes at least two of the three major sub-populations of leucocytes selected from the group consisting of monocytes, granulocytes and lymphocytes;
determining the fluorescence intensity for each of the identified sub-populations using a single fluorescence from the stained nucleic acid within the cells; and
calculating a ratio of the fluorescence intensity of one cell sub-population to the fluorescence intensity of another cell sub-population.

45. An assay according to claim 44, wherein the sample of leucocytes is from a blood sample of the patient or from cultured leucocytes.

46. An assay according to claim 44, wherein the monocyte, granulocyte and lymphocyte sub-populations are all identified.

47. An assay according to claim 44, wherein the fluorescence intensity of each sub-population is determined from the mean or median fluorescence intensity of the respective sub-population.

48. An assay according to claim 44, wherein at least one of the following ratios is calculated:
monocytes:granulocytes
monocytes:lymphocytes; and
granulocytes:lymphocytes.

49. An assay according to claim 44, wherein the viral infection is HIV and/or the disease is AIDS.

50. An assay according to claim 44, which additionally monitors co-infection of the patient with another disease.

51. An assay according to claim 50, which indicates a co-infection when the ratio of the mean fluorescence intensity of the monocyte population to the mean fluorescence intensity of the granulocyte population is less than the ratio of the mean fluorescence intensity of the monocyte population to the mean fluorescence intensity of the lymphocyte population.

52. An assay according to claim 44, further comprising a step for obtaining a CD4 count.

53. An assay according to claim 52, wherein the CD4 count is obtained by adding to the sample an antibody that fluoresces in a different fluorescent channel to the dye.

54. A kit for diagnosing or monitoring a viral, parasitic or bacterial infection or disease in a patient comprising a cell membrane-permeable dye which stains RNA.

55. A kit according to claim 54, wherein the cell membrane-permeable dye stains RNA in a single fluorescence.

56. A kit according to claim 54, further comprising computer readable instructions for performing an assay comprising at least one of the following steps:
identifying at least two sub-populations in a sample of leucocytes;
calculating a fluorescence intensity of each identified sub-population; and
calculating a ratio of the fluorescence intensity of one sub-population to another using a single fluorescence of a stained nucleic acid within the cells.

57. A kit according to claim 56, wherein the sub-populations are selected from the group consisting of monocytes, granulocytes and lymphocytes.

58. A kit according to claim 56, wherein the computer readable instructions interpret the obtained ratio or ratios to indicate whether the patient has a low, medium or high viral, parasitic or bacterial reservoir or has a co-infection.

59. A kit according to claim 54, further comprising an antibody for determining a CD4 count of a sample of leucocytes.

60. A kit according to claim 54, further comprising cell membrane markers or intracellular markers for phenotyping.

61. A kit according to claim 60, wherein the markers are selected from the group consisting of CD38, CD14/CD16 and p24.

62. A kit according to claim 54, further comprising one or more reagents selected from the group consisting of a red cell lysing agent, a stabilizer, a fixative, control cells, media and bead reagents.

63. A kit according to claim 62, further comprising means for dispensing the red cell lysing agent, dye, antibody reagents and/or other reagents used in the assay.

64. A machine readable medium comprising instructions for diagnosing or monitoring the cellular viral, parasitic or bacterial reservoir of a patient wherein the machine performs at least one of the following steps:
mixing a sample of leucocytes with a fluorescent cell membrane-permeable dye which stains RNA within the leucocytes;
identifying from all the leucocytes at least two of the three major sub-populations of leucocytes selected from the group consisting of monocytes, granulocytes and lymphocytes;
determining the fluorescence intensity for each of the identified sub-populations using a single fluorescence from the stained nucleic acid within the cells; and
calculating a ratio of the fluorescence intensity of one cell sub-population to the fluorescence intensity of another cell sub-population.

65. A machine readable medium according to claim 64, which is configured for use in conjunction with a flow cytometer and/or haematology analyser.

66. A machine readable medium according to claim 64, further comprising instructions for performing analysis methods selected from the group consisting of impedance, light scatter and fluorescence.
